(19) United States
(12) Patent Application Publication
(10) Pub. No.: US 2019/0083067 A1
(43) Pub. Date: Mar. 21, 2019

(54) METHODS AND SYSTEMS FOR CORRECTION OF ONE DIMENSIONAL SHEAR WAVE DATA

(71) Applicant: General Electric Company, Schenectady, NY (US)

(72) Inventor: Jeong Seok Kim, Seongnam-si (KR)

(21) Appl. No.: 15/711,329

(22) Filed: Sep. 21, 2017

(51) Int. Cl. A61B 8/08; A61B 8/14; G01S 7/52; G01S 15/89

(52) U.S. Cl. CPC A61B 8/5207; A61B 8/485; A61B 8/5223; G01S 15/8979; A61B 8/488; A61B 8/14; G01S 7/52042; A61B 8/085

(57) ABSTRACT

Methods and systems relate to correcting one-dimensional (1D) shear wave data of an anatomical structure. The methods and systems obtaining a 1D shear wave data from an ultrasound probe of the anatomical structure. The methods and systems adjust a velocity or a pressure of the 1D shear wave data based on the correction model to form adjusted 1D shear wave data. The adjustment of the velocity or the pressure of the 1D shear wave data is based on weights applied to the 1D shear wave data from hidden layers of the correction model. The methods and systems generate a 1D shear wave image based on the adjusted 1D shear wave data.

### FIELD

Embodiments described herein generally relate to correcting one-dimensional shear wave data of an anatomical structure.

### BACKGROUND OF THE INVENTION

During an ultrasound exam a shear wave may be generated to measure a shear elasticity of an anatomical structure. For example, the shear elasticity of a liver provides a degree of liver stiffness measured by the shear wave, which can determine liver fibrosis and/or cirrhosis. The shear elasticity can be determined based on one-dimensional (1D) shear wave data acquired by a conventional ultrasound imaging system. However, the 1D shear wave data is dependent on densities of the anatomical structure. For example, the 1D shear wave data is highly dependent on a thickness of fat and/or muscle within the anatomical structure, which affects pressure and/or speed measurements of the 1D shear wave data.

### BRIEF DESCRIPTION OF THE INVENTION

In an embodiment a method (e.g., for correcting one-dimensional shear wave data of an anatomical structure) is provided. The method includes obtaining a 1D shear wave data from an ultrasound probe of the anatomical structure. The method includes adjusting a velocity or a pressure of the 1D shear wave data based on the correction model to form adjusted 1D shear wave data. The adjustment of the velocity or the pressure of the 1D shear wave data is based on weights applied to the 1D shear wave data from hidden layers of the correction model. The method includes generating a 1D shear wave image based on the adjusted 1D shear wave data.

In an embodiment a system (e.g., medical imaging system) is provided. The system includes an ultrasound probe configured to acquire 1D shear wave data of an anatomical structure, a communication circuit configured to receive two-dimensional (2D) shear wave data along a communication link from a remote server, and a display. The system includes a controller circuit. The controller circuit is configured to obtain the 1D shear wave data from an ultrasound probe of the anatomical structure. The controller circuit is configured to adjust a velocity or a pressure of the 1D shear wave data based on the correction model to form adjusted 1D shear wave data. The adjustment of the velocity or the pressure of the 1D shear wave data is based on weights applied to the 1D shear wave data from hidden layers of the correction model. The controller circuit is configured to generate a 1D shear wave image based on the adjusted 1D shear wave data on the display.

In an embodiment a tangible and non-transitory computer readable medium is provided, which includes one or more programmed instructions configured to direct one or more processors. The one or more processors are instructed to obtain a 1D shear wave data from an ultrasound probe of the anatomical structure. The one or more processors are instructed to adjust a velocity or a pressure of the 1D shear wave data based on the correction model to form adjusted 1D shear wave data. The adjustment of the velocity or the pressure of the 1D shear wave data is based on weights applied to the 1D shear wave data from hidden layers of the correction model. The one or more processors are instructed to generate a 1D shear wave image based on the adjusted 1D shear wave data.

### BRIEF DESCRIPTION OF THE DRAWINGS

FIG. 1 illustrates a schematic block diagram of an embodiment of a medical imaging system.

FIG. 2 illustrates an embodiment of a one-dimensional shear wave model.

FIG. 3 illustrates a flow chart of an embodiment of a method for correcting one-dimensional shear wave data of an anatomical structure.

FIG. 4 illustrates an embodiment of two-dimensional shear wave data.

FIG. 5 illustrates an embodiment for acquisition of one-dimensional shear wave data.

FIG. 6 illustrates an embodiment for generating a one-dimensional shear wave image.

### DETAILED DESCRIPTION OF THE INVENTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments described herein generally relate to correcting one-dimensional (1D) shear wave data of an anatomical structure. The correcting of the 1D shear wave data is based on a correction model. The correction model represents a machine learning algorithm trained and/or configured based on two-dimensional (2D) shear wave data of the anatomical structure. The characteristics of a shear-wave for 2D shear wave data is produced using an ultrafast sequence (e.g., up to 20,000 Hz) of vibrations produced by an ultrasound probe to generate the shear waves along 2Ds. The 2D shear wave data includes information corresponding to anatomical features of the anatomical structure, such as fat and muscle. For example, the 2D shear wave data includes data on a shear-wave speed and/or pressure applied to the anatomical structure passing through the anatomical structure, which provides data on underlying tissue of the anatomical structure. The information relating to the anatomical feature allow the correction model to adjust the 1D shear wave data.

The correction model is based on a plurality and/or multiple sets of 2D shear wave data from one or more patients. The correction model may be received by the ultrasound imaging system from a remote server and/or generated by the medical imaging system from the received sets of 2D shear wave data. The correction model represents a machine learning algorithm. For example, the correction model may represent an artificial neural network. The correction model may include a plurality of artificial neural layers, such as an input layer, one or more hidden layers, and an output layer. The correction model is configured to be trained based on portions of the anatomical structure measured by the sets of 2D shear wave data. For example, the anatomical structure is subdivided into different portions corresponding to different anatomical features of the anatomical structure. The correction model receives the portions at the input layer. The portions represent different characteristics and/or densities (e.g., fat, muscle) of the anatomical structure. The correction model is trained to define weights at the hidden layers. The hidden layers adjust pixels of the 1D shear wave data representing pressure and/or speed based on the characteristics of the 1D shear wave data.

Optionally, the correction model may include a Stacked Denoise Autoencoder architecture (SDAA). The SDAA is configured to continually adjust weights applied at the hidden layers of the correction model. The adjustment of the weights is based on a backpropagation error and/or gradient. The gradient is calculated based on a mean of the 2D shear wave data and the adjusted 1D shear wave data outputted by the correction model. The weights of the hidden layers are adjusted based on the gradient. Additionally or alternatively, the SDAA is configured to improve the 1D shear wave data, which can include noise. By continually adjusting the weights based on the 2D shear wave data, the SDAA minimizes the affect of the noise of the 1D shear wave data.

A technical effect of at least one embodiment described herein provides an accurate measurement values for 1D shear wave data. A technical effect of at least one embodiment described herein enables a user to receive accurate diagnostic value of a patient independent of an abdominal shape of the patient.

### Terms

The term “one-dimensional shear wave data” and/or “1D shear wave data” refers to ultrasound data that is acquired by vibrating an ultrasound probe on a surface of a patient. The vibrations represent a mechanical force applied to the surface of the patient, which generates one or more shear waves. The shear wave can extend 25-65 centimeters into the anatomical structure. The 1D shear wave data represents a speed of the shear wave traversing through the anatomical structure. For example, the ultrasound probe acquires ultrasound data corresponding to an M-mode (e.g., ultrasound imaging mode). The medical imaging system tracks the shear wave propagation through the anatomical structure by measuring tissue displacement. Additionally or alternatively, based on the speed of the shear wave a pressure (e.g., kPa) can be determined using the Young's modulus.

The term “two-dimensional shear wave data” and/or “2D shear wave data” refers to ultrasound data that is acquired by applying a series of pulses and/or a quasi-static pulse occurring at a push location on a surface of a patient. The series of pulses and/or the quasi-static pulses generate one or more shear waves traversing through the anatomical structure within the patient. The series of pulses are generated by a mechanical force oscillating on an ultrasound probe. For example, the series of pulses can be generated by an electric motor mechanically coupled to the ultrasound probe. The series of pulses may be periodic having a fixed frequency and/or time-varying by shifting between a range of frequencies. The quasi-static pulse is applied by a clinician applying a force and/or compression of the ultrasound probe to the surface of the patient. The 2D shear wave data represents a speed of the shear wave traversing through the anatomical structure based on a comparison of ultrasound images as the shear wave traverses through the anatomical structure. For example, the medical imaging system compares two ultrasound images (e.g., B-mode ultrasound images, plane wave). The first ultrasound image represents the anatomical image prior to the application of the shear wave. The second ultrasound image represents the anatomical image as the shear wave traverses through the anatomical structure. Based on a different in shape and/or size of the anatomical structure between the two ultrasound images, the Young's modulus is determined. The Young's modulus is utilized by the medical imaging system to determine a pressure applied to the anatomical structure based on the shear wave.

The term “anatomical structure” refers to an anatomical part of a patient. Non-limiting example of an anatomical structure includes an organ (e.g., heart, kidney, lung, liver, bladder, brain, neonatal brain, embryo, abdomen, and/or the like), vascular structure (e.g., vein, artery, mitral valve, aortic valve, tricuspid valve, pulmonary valve), tissue or portion of an organ (e.g., left ventricular apex, right ventricular outflow-track, intra-ventricular septum, breast tissue, liver tissue, brain tissue, cardiac tissue, prostate tissue, and/or the like), skeletal structure, and/or the like.

The term “anatomical feature” refers to a structural feature of the anatomical structure. Non-limiting examples of anatomical features include dimensions (e.g., height, length, width, depth), a shape, a boundary dimension (e.g., thickness, shape), density (e.g., fat, muscle), a number of cavities or chambers, fiducial markers, and/or the like.

The term “characteristic vector” refers to a vector indicative of a pressure applied to an anatomical structure and/or a speed of a shear wave traversing through the anatomical structure acquired by 1D and/or 2D shear wave data. The pressure and/or speed are indicated by a pixel (e.g., color, intensity) of the 1D and/or 2D shear wave data. The characteristic vector may represent one or more pixel characteristics of the 1D and/or 2D shear wave data. For example, the characteristic vector can include values for one or more of an intensity, a color, a gradient, a histogram, and/or the like of the pixel.

The term “real time” or “real-time” is used to refer to an operation, action, and/or process performed by the medical imaging system (e.g., a controller circuit) during an ultrasound exam. An ultrasound exam may include collection of multiple separate 2D, 3D, and/or 4D ultrasound images for a common or different view windows. Optionally, the ultrasound exam may include collection of one or more cine loops of 2D or 3D ultrasound data. The operation, action or process may be performed while actively scanning a patient and/or between separate scanning operations that occur during a single ultrasound exam. A length of time associated with real time, and may vary based on a processing speed and/or operating specification (e.g., no intentional lag or delay). Real time includes updating an ultrasound image shown on the display after each ultrasound pulse within a scan and/or after each ultrasound scan sequence. Additionally or alternatively, ultrasound data may be stored temporarily in memory of the medical imaging system during the ultrasound exam and processed in a live or off-line operation.

The term “machine learning algorithm” refers to an artificial intelligence algorithm that learns from various automatic or manual inputs, such as observations and/or data. The machine learning algorithm is adjusted over multiple iterations based on the observations and/or data. For example, the machine learning algorithm is adjusted by supervised learning, unsupervised learning, and/or reinforcement learning. Non-limiting examples of machine learning algorithms are a decision tree, K-means, deep learning, artificial neural network, and/or the like.

The term “correction model” refers to a machine learning algorithm that has been trained based on 2D shear wave data to identify anatomical features (e.g., fat, muscle) of an anatomical structure within an anatomical structure. The correction model utilizes the 2D shear wave data to determine adjustments to pressure and/or speed measurements. The correction model receives 1D shear wave data from the medical imaging system. The pixels of the 1D shear wave data represent different pressure and/or speed measurements. The correction model identifies the anatomical features (e.g., fat, muscle) of the pixels, and adjusts the pixels to form the adjusted 1D shear wave data. The adjustment to the pixels is based on the anatomical features represented by the pixels. For example, the correction model reduces a speed represented by the pixels when the anatomical feature of the pixel is fat.

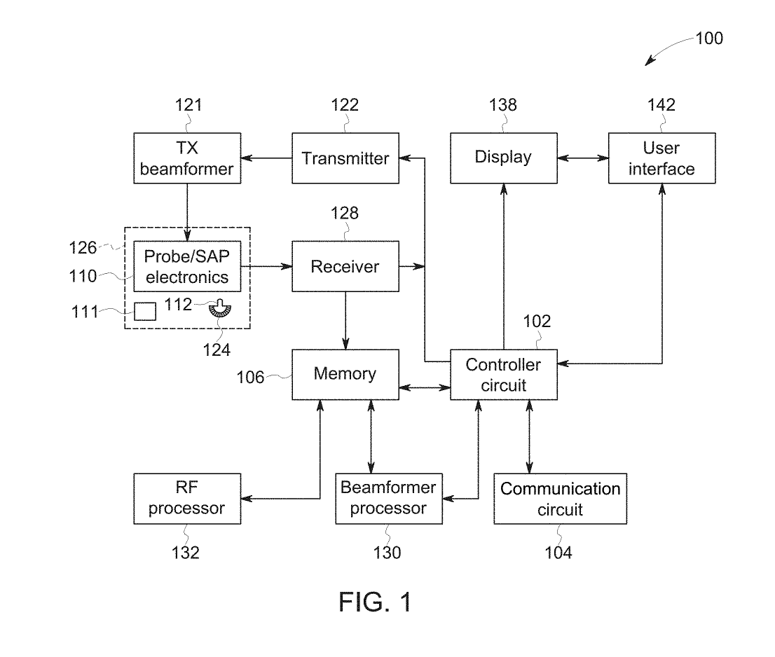

FIG. 1 illustrates a schematic block diagram of an embodiment of a medical imaging system 100. For example, the medical imaging system 100 is shown as an ultrasound imaging system. The medical imaging system 100 may include a controller circuit 102 operably coupled to a communication circuit 104, a display 138, a user interface 142, an ultrasound probe 126, and a memory 106.

The controller circuit 102 is configured to control the operation of the medical imaging system 100. The controller circuit 102 may include one or more processors. Optionally, the controller circuit 102 may include a central processing unit (CPU), one or more microprocessors, a graphics processing unit (GPU), or any other electronic component capable of processing inputted data according to specific logical instructions. Optionally, the controller circuit 102 may include and/or represent one or more hardware circuits or circuitry that include, are connected with, or that both include and are connected with one or more processors, controllers, and/or other hardware logic-based devices. Additionally or alternatively, the controller circuit 102 may execute instructions stored on a tangible and non-transitory computer readable medium (e.g., the memory 106).

The controller circuit 102 is configured to adjust obtained 1D shear wave data representing the anatomical structure based on a correction model. The anatomical features can represent a density of the anatomical structure, such as fat or muscle. For example, the controller circuit 102 executes the correction model, which adjusts pixels representing pressure and/or velocity measurements of the 1D shear wave data. The 1D shear wave data can be acquired during an ultrasound exam in real time. Optionally, the 1D shear wave data may be accessed by the controller circuit 102 in the memory 106 and/or received from a remote server.

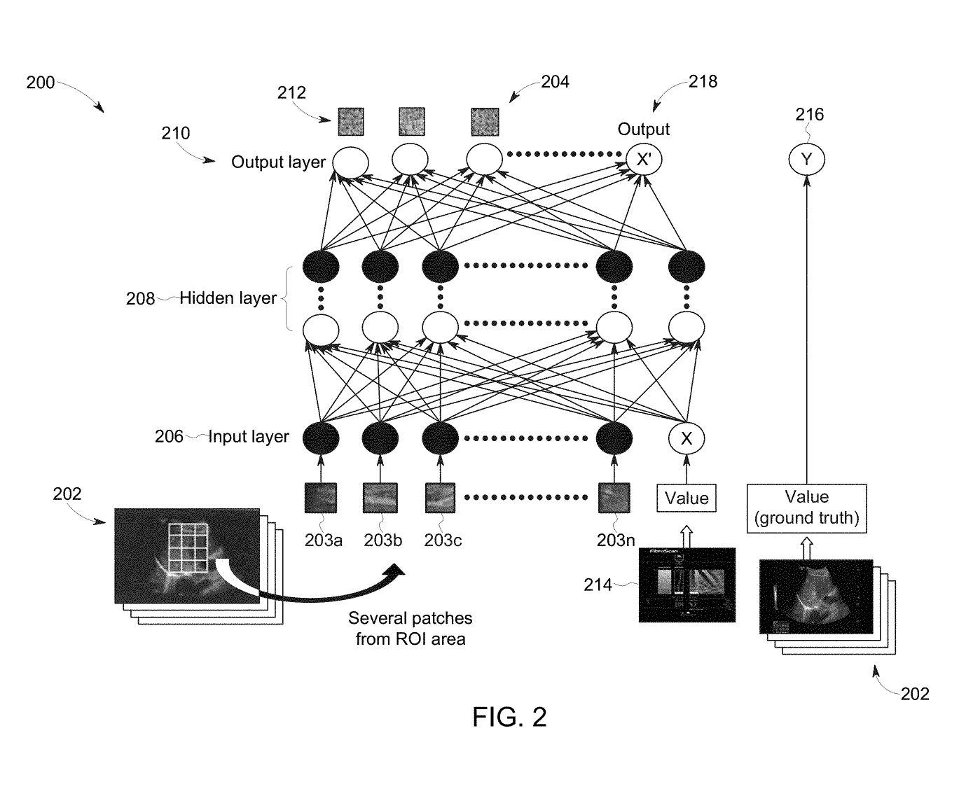

The correction model represents one or more machine learning algorithms trained and/or configured based on sets of 2D shear wave data. The correction model can be stored in the memory 106. Optionally, the controller circuit 102 may receive the correction model from the remote server and/or an alternative medical imaging system via the communication circuit 104. The correction model may be executed by the controller circuit 102 as the 1D shear wave data are being acquired (e.g., in real-time) by the medical imaging system 100. Additionally or alternatively, the correction model is executed by the controller circuit 102 as the 1D shear wave data are loaded by the clinician from the memory 106, the remote server and/or the alternative medical imaging system. In connection with FIG. 2, the correction model 204 is configured based on sets of 2D shear wave data 202.

FIG. 2 illustrates an embodiment 200 of the correction model 204. The correction model 204 is shown as an artificial neural network. For example, the correction model 204 includes an input layer 206, one or more hidden layers 208, and an output layer 210. The correction model 204 may be trained by the controller circuit 102 and/or received by the controller circuit 102 from the remote server via the communication circuit 104. It may be noted that the correction model 204 may represent alternative machine learning algorithms. For example, the correction model 204 may represent a classification algorithm, a deep learning algorithm, a clustering algorithm, and/or the like.

The artificial neural network is trained based on the sets of 2D shear wave data 202. The sets of 2D shear wave data 202 may have been acquired from one or more patients. For example, the sets of 2D shear wave data 202 may be received from the remote server via the communication circuit 104. Additionally or alternatively, the sets of 2D shear wave data 202 may be acquired from the ultrasound probe 126. The sets of 2D shear wave data is subdivided by the controller circuit 102 into patches and/or portions 203a-n. The portions 203a-n represent different anatomical features, such as different densities of the anatomical structure. For example, a majority of the portion 203a can represent fat and a majority of the portion 203c may represent muscle. The portions 203a-n are received at the input layer 206 and transformed by the controller circuit 102 into characteristic vectors.

The characteristic vectors may represent information that is indicative of the pixels of the portions 203a-n. For example, the pixels can indicate a pressure of the anatomical structure as the shear wave traverses through the anatomical structure and/or a speed of the shear wave passing through the anatomical structure. The characteristic vectors are a representation of the pixel information within the portions 203a-n. For example, the characteristic vectors can include a histogram, gradients, a mean color, an intensity or brightness, and/or the like of the pixels of the portions 203a-n. Based on the characteristic vectors, the input layer 206 directs the portions 203a-n to corresponding hidden layers 208. For example, the characteristic vectors may represent a histogram of the portions 203a-n representing a velocity based on the pixel characteristics. The changes in the velocity can be identified by the controller circuit 102 representing different anatomical features within the portions 203a-n. For example, the controller circuit 102 may identify an increase in the speed of the shear wave form through one of the portions 203a-n. The controller circuit 102 determines the increase in velocity corresponds to a high fat content. The controller circuit 102 may instruct the input layer 206 to direct the corresponding characteristic vectors of the one of the portions 203a-n having the high fat content to a first portion of the hidden layers 208.

Additionally or alternatively, the controller circuit 102 may identify a decrease in the velocity of the shear wave form through one of the portions 203a-n. The controller circuit 102 determines the decrease in velocity corresponds to a high muscle content. The controller circuit 102 may instruct the input layer 206 to direct the corresponding characteristic vectors of the one of the portions 203a-n having the high muscle content to a second portion of the hidden layers 208. By the controller circuit 102 directing the portions 203a-n to specific hidden layers 208, the hidden layers 208 are configured having defined weight matrixes to specific anatomical features (e.g., fat, muscle).

The one or more hidden layers 208 represent different functions and/or outputs of the correction model 204. The hidden layers 208 represent different groups or sets of artificial neurons, which represent functions performed by the controller circuit 102 on the characteristic vectors. The artificial neurons of the hidden layers 208 adjust the characteristic vectors of 1D shear wave data based on the weight matrixes. For example, each of the hidden layers 208 may represent a weight matrix and an offset vector and/or bias.

The weight matrixes represent predetermined values configured based on the portions 203a-n of the sets of 2D shear wave data 202. For example, during the training of the correction model 204 the weight matrixes are defined based on the portions 203a-n. For example, the weight matrix of the hidden layers 208 adjusts characteristic vectors, which represent a majority of fat. The hidden layers 208 adjust the characteristic vectors representing fat of the portion 203a such that the fat content does not artificially increase a speed of the shear wave traversing through the anatomical structure. The output layer 210 outputs 212 the portions 203a-n representing different anatomical features received from the hidden layers 208.

The controller circuit 102 acquires the 1D shear wave data 214 from the ultrasound probe 126. For example, the controller circuit 102 may instruct a shear wave generator 111 of the ultrasound probe 126 to generate a transient impulse on a surface of the patient. As the shear wave from the transient impulse traverses through the anatomical structure, the transducer array 112 receives the 1D swear wave data 214 representing tissue displacement of the anatomical structure based on the shear wave. The 1D shear wave data 214 are received at the input layer 206 and transformed by the controller circuit 102 into the characteristic vectors. The characteristic vectors may represent an array of information that is indicative of the pixels of the 1D shear wave data 214, which represents the pressure applied to the anatomical structure and/or the speed of the shear wave traversing through the anatomical structure. The 1D shear wave data 214 is received by the hidden layers 208 from the input layer 206. The controller circuit 102 identifies portions of the characteristic vectors that correspond to different anatomical features. For example, the controller circuit 102 identifies changes in speed and/or pressure of the shear wave of the characteristic vectors corresponding to different anatomical features. The controller circuit 102 instructs the characteristic vectors corresponding to the fat content to the first portion of the character vector to hidden layers 208 corresponding to weight matrixes for fat content. The hidden layers 208 apply weights to the characteristic vectors based on the weights matrixes of the 1D shear wave data 214.

The hidden layers 208 adjust the characteristic vectors of the 1D shear wave data 214. For example, the hidden layers 208 adjust the pixel intensities of the characteristic vectors, which adjust the velocity and/or pressure of the shear wave measured by the 1D shear wave data 214. The adjustment of the pixel intensities are configured to adjust portions of the 1D shear wave data 214 that is indicative of a density (e.g., anatomical features) of the anatomical structure. For example, the hidden layers 208 reduce the velocity of the shear wave traversing through content of the anatomical structure representing fat. In another example, the hidden layers 208 increase the velocity of the shear waves traversing through content of the anatomical structure representing muscle.

The output layer 210 outputs an adjusted 1D shear wave data 218. The adjusted 1D shear wave data 218 includes the adjusted characteristic vectors based on the hidden layers 208. The correction model 204 applies different weights to portions of the 1D shear wave data 214, which adjusts a measured velocity and/or pressure of the shear wave traversing through the anatomical structure. For example, the adjusted 1D shear wave data 218 includes at least an adjusted velocity and/or pressure measurement based on the weights of the hidden layers 208.

Optionally, the correction model 204 may include a Stacked Denoise Autoencoder architecture (SDAA). The SDAA is configured to adjust the weight matrix values of the hidden layers 208 by comparing the sets of 2D shear wave data 202 with the adjusted 1D shear wave data 218. For example, the controller circuit 102 calculates a mean 216 of the pressure and/or speed for the sets of 2D shear wave data 202 as an optimal and/or true data. The optimal and/or true data is treated by the controller circuit 102 as a control and/or default measured values of the speed and/or pressure. The controller circuit 102 compares the mean 216 with the adjusted 1D shear wave data 218 as shown in Equation 1. For example, the mean 216 is adjusted by the adjusted 1D shear wave data 218.

$$\frac{dE}{dw} = ( Y - {X}^{′} ) ( Y - {X}^{T} ) \qquad Equation ( 1 )$$

Equation 1 represents an output error and/or loss with respect to the weight matrixes of the hidden layers 208. The output error is shown as the variable E. The output error is indicative of a difference and/or deviation between the mean 216 and the adjusted 1D shear wave data 218. The variable Y represents the mean 216. The variable X′ represents the adjusted 1D shear wave data 218. The variable X^{T} represents the transpose of the adjusted 1D shear wave data 218 and/or characteristic vectors. The variable w represents the weight matrixes of the hidden layers 208. The result of Equation 1 represents a rate of the output error and the weight.

Based on the rate calculated in Equation 1, the controller circuit 102 utilizes Equation 2 to adjust the weight matrixes of the hidden layers 208. Equation 2 is configured to reduce a backpropagation of the correction model 204. The backpropagation represents an error contribution of at least one of the hidden layers 208 to the adjusted 1D shear wave data 218. For example, Equation 2 identifies a gradient of the output error, which is represented as the variable Δw. The variable η represents the learning rate for the 1D swear wave model 204. The learning rate adjusts the weight of the rate. The controller circuit 102 adjusts the hidden layers 208 based on the gradient, which distributes the adjustment across the hidden layers 208 of the 1D swear wave model 204.

$$w = {w}_{0} + Δ w , Δ w = - η \cdot \frac{dE}{dw} \qquad Equation ( 2 )$$

For example, the variable w₀ represents the current weight matrixes of the hidden layers 208. The controller circuit 102 adjusts the magnitude of the weight matrixes by the gradient as shown in Equation 2 to form the adjusted weight matrixes, shown as the variable w.

The controller circuit 102 (FIG. 1) may be operably coupled to and/or control the communication circuit 104. The communication circuit 104 is configured to receive and/or transmit information with one or more alternative medical imaging systems, the remote server, and/or the like along a uni-directional and/or bi-directional communication link. The remote server may represent a database that includes patient information, machine learning algorithms, remotely stored ultrasound images of a patient, and/or the like. The communication circuit 104 may represent hardware that is used to transmit and/or receive data along the uni-directional and/or bi-directional communication link. The communication circuit 104 may include a transceiver, receiver, transceiver and/or the like and associated circuitry (e.g., antennas) for wired and/or wirelessly communicating (e.g., transmitting and/or receiving) with the one or more alternative medical imaging systems, the remote server, and/or the like. For example, protocol firmware for transmitting and/or receiving data along the uni-directional and/or bi-directional communication link may be stored in the memory 106, which is accessed by the controller circuit 102. The protocol firmware provides the network protocol syntax for the controller circuit 102 to assemble data packets, establish and/or partition data received along the bi-directional communication links, and/or the like.

The uni-directional and/or bi-directional communication links may be a wired (e.g., via a physical conductor) and/or wireless communication (e.g., utilizing radio frequency (RF)) link for exchanging data (e.g., data packets) between the one or more alternative medical imaging systems, the remote server, and/or the like. The bi-directional communication links may be based on a customized communication protocol and/or a standard communication protocol, such as Ethernet, TCP/IP, Wi-Fi, 802.11, Bluetooth, and/or the like.

The controller circuit 102 is operably coupled to the display 138 and the user interface 142. The display 138 may include one or more liquid crystal displays (e.g., light emitting diode (LED) backlight), organic light emitting diode (OLED) displays, plasma displays, CRT displays, and/or the like. The display 138 may display patient information, one or more ultrasound images and/or videos, components of a graphical user interface, one or more 2D, 3D, or 4D ultrasound image data sets from ultrasound data stored in the memory 106 or currently being acquired in real-time, anatomical measurements, diagnosis, treatment information, tags, and/or the like received by the display 138 from the controller circuit 102.

The user interface 142 controls operations of the controller circuit 102 and the medical imaging system 100. The user interface 142 is configured to receive inputs from the clinician and/or operator of the medical imaging system 100. The user interface 142 may include a keyboard, a mouse, a touchpad, one or more physical buttons, and/or the like. Optionally, the display 138 may be a touch screen display, which includes at least a portion of the user interface 142. For example, a portion of the user interface 142 may correspond to a graphical user interface (GUI) generated by the controller circuit 102, which is shown on the display 138. The touch screen display can detect a presence of a touch from the operator on the display 138 and can also identify a location of the touch with respect to a surface area of the display 138. For example, the user may select one or more user interface components of the GUI shown on the display by touching or making contact with the display 138. The user interface components may correspond to graphical icons, textual boxes, menu bars, and/or the like shown on the display 138. The user interface components may be selected, manipulated, utilized, interacted with, and/or the like by the clinician to instruct the controller circuit 102 to perform one or more operations as described herein. The touch may be applied by, for example, at least one of an individual's hand, glove, stylus, and/or the like.

The memory 106 includes parameters, algorithms, protocols of one or more ultrasound exams, data values, and/or the like utilized by the controller circuit 102 to perform one or more operations described herein. The memory 106 may be a tangible and non-transitory computer readable medium such as flash memory, RAM, ROM, EEPROM, and/or the like.

The memory 106 may include the 1D swear wave model 204. The controller circuit 102 executes the 1D swear wave model 204 to adjust the 1D shear wave data 214 acquired by the medical imaging system 100. Optionally, the 1D swear wave model 204 may be received along the uni-directional or bi-directional communication links via the communication circuit 104 and stored in the memory 106.

The ultrasound probe 126 may have a transmitter 122, transmit beamformer 121 and probe/SAP electronics 110. The probe/SAP electronics 110 may be used to control the switching of the transducer elements 124. The probe/SAP electronics 110 may also be used to group transducer elements 124 into one or more sub-apertures. The ultrasound probe 126 may be configured to acquire ultrasound data or information from the anatomical structure of the patient. The ultrasound probe 126 is communicatively coupled to the controller circuit 102 via the transmitter 122. The transmitter 122 transmits a signal to a transmit beamformer 121 based on acquisition settings received by the controller circuit 102. The acquisition settings may define an amplitude, pulse width, frequency, gain setting, scan angle, power, time gain compensation (TGC), resolution, and/or the like of the ultrasonic pulses emitted by the transducer elements 124. The transducer elements 124 emit pulsed ultrasonic signals into the patient (e.g., a body). The acquisition settings may be defined by the user operating the user interface 142. The signal transmitted by the transmitter 122 in turn drives a plurality of transducer elements 124 within a transducer array 112.

The transducer elements 124 emit pulsed ultrasonic signals into a body (e.g., patient) or volume corresponding to the acquisition settings along one or more scan planes. The ultrasonic signals may include, for example, one or more reference pulses, imaging pulses, one or more pushing pulses (e.g., shear-waves), and/or one or more pulsed wave Doppler pulses. At least a portion of the pulsed ultrasonic signals backscatter from the anatomical structure to produce echoes. The echoes are delayed in time and/or frequency according to a depth or movement, and are received by the transducer elements 124 within the transducer array 112. The ultrasonic signals may be used for imaging, for generating and/or tracking shear-waves, for measuring changes in position or velocity within the anatomic structure, differences in compression displacement of the tissue (e.g., strain), and/or for therapy, among other uses. For example, the probe 126 may deliver low energy pulses during imaging and tracking, medium to high energy pulses to generate shear-waves, and high energy pulses during therapy.

The transducer elements 124 convert the received echo signals into electrical signals, which may be received by a receiver 128. The receiver 128 may include one or more amplifiers, an analog to digital converter (ADC), and/or the like. The receiver 128 may be configured to amplify the received echo signals after proper gain compensation and convert these received analog signals from each transducer element 124 to digitized signals sampled uniformly in time. The digitized signals representing the received echoes are stored in memory 106, temporarily. The digitized signals correspond to the backscattered waves received by each transducer element 124 at various times. After digitization, the signals still may preserve the amplitude, frequency, phase information of the backscatter waves.

Optionally, the controller circuit 102 may retrieve the digitized signals stored in the memory 106 to prepare for the beamformer processor 130. For example, the controller circuit 102 may convert the digitized signals to baseband signals or compressing the digitized signals.

The ultrasound probe 126 includes the shear wave generator 111. The shear wave generator 111 may represent an electric motor that is operably coupled to the ultrasound probe 126. The electric motor may include a weighted offset shaft, which is configured to vibrate the electric motor. For example, the ultrasound probe 126 is positioned adjacent to and/or on a skin of a patient. When the shear wave generator 111 is activated, the weighted offset shaft rotates generating vibrations to form a shear wave (e.g., for 1D shear wave data 214). The controller circuit 102 may form a transient impulse and/or a series of pulses (e.g., for 2D shear wave data) based on a rotation of the weighted offset shaft of the electric motor. For example, to form the transient impulse the controller circuit 102 activates the shear wave generator 111 by generating one or more pulses, which is received by the shear wave generator 111. The one or more pulses may have a pulse width of 20 nanoseconds to form the transient impulse. In another example, to form the series of pulses the controller circuit 102 may activate the shear wave generator 111 by generating one or more pulses, which is received by the shear wave generator 111. The one or more pulses may have a fixed frequency and/or a time-varying frequency (e.g., changing frequency).

The beamformer processor 130 may include one or more processors. Optionally, the beamformer processor 130 may include a central processing unit (CPU), one or more microprocessors, or any other electronic component capable of processing inputted data according to specific logical instructions. Additionally or alternatively, the beamformer processor 130 may execute instructions stored on a tangible and non-transitory computer readable medium (e.g., the memory 106) for beamforming calculations using any suitable beamforming method such as adaptive beamforming, synthetic transmit focus, aberration correction, synthetic aperture, clutter reduction and/or adaptive noise control, and/or the like. Optionally, the beamformer processor 130 may be integrated with and/or a part of the controller circuit 102. For example, the operations described as being performed by the beamformer processor 130 may be configured to be performed by the controller circuit 102.

The beamformer processor 130 performs beamforming on the digitized signals of transducer elements and outputs a radio frequency (RF) signal. The RF signal is then provided to an RF processor 132 that processes the RF signal. The RF processor 132 may include one or more processors. Optionally, the RF processor 132 may include a central processing unit (CPU), one or more microprocessors, or any other electronic component capable of processing inputted data according to specific logical instructions. Additionally or alternatively, the RF processor 132 may execute instructions stored on a tangible and non-transitory computer readable medium (e.g., the memory 106). Optionally, the RF processor 132 may be integrated with and/or a part of the controller circuit 102. For example, the operations described as being performed by the RF processor 132 may be configured to be performed by the controller circuit 102.

The RF processor 132 may generate different ultrasound image data types and/or modes (e.g., shear wave/elasticity mode, B-mode, C-mode, M-mode, color Doppler (e.g., color flow, velocity/power/variance), tissue Doppler, and Doppler energy) for multiple scan planes or different scanning patterns based on the predetermined settings of the first model. For example, the RF processor 132 may generate tissue Doppler data for multi-scan planes. The RF processor 132 gathers the information (e.g., I/Q, B-mode, color Doppler, tissue Doppler, shear wave/elasticity, and Doppler energy information) related to multiple data slices and stores the data information, which may include time stamp and orientation/rotation information, in the memory 106.

Alternatively, the RF processor 132 may include a complex demodulator (not shown) that demodulates the RF signal to form IQ data pairs representative of the echo signals. The RF or IQ signal data may then be provided directly to the memory 106 for storage (e.g., temporary storage). Optionally, the output of the beamformer processor 130 may be passed directly to the controller circuit 102.

The controller circuit 102 may be configured to process the acquired ultrasound data (e.g., RF signal data or IQ data pairs) and prepare and/or generate frames of ultrasound image data representing the anatomical structure for display on the display 138. Acquired ultrasound data may be processed in real-time by the controller circuit 102 during the ultrasound exam as the echo signals are received. Additionally or alternatively, the ultrasound data may be stored temporarily in the memory 106 during the ultrasound exam and processed in less than real-time in a live or off-line operation.

The memory 106 may be used for storing processed frames of acquired ultrasound data that are not scheduled to be displayed immediately or to store post-processed images, firmware or software corresponding to, for example, a graphical user interface, one or more default image display settings, programmed instructions, and/or the like. The memory 106 may store the ultrasound images such as 3D ultrasound image data sets of the ultrasound data, where such 3D ultrasound image data sets are accessed to present 2D, 3D, and/or 4D images. For example, a 3D ultrasound image data set may be mapped into the corresponding memory 106, as well as one or more reference planes. The processing of the ultrasound data, including the ultrasound image data sets, may be based in part on user inputs, for example, user selections received at the user interface 142.

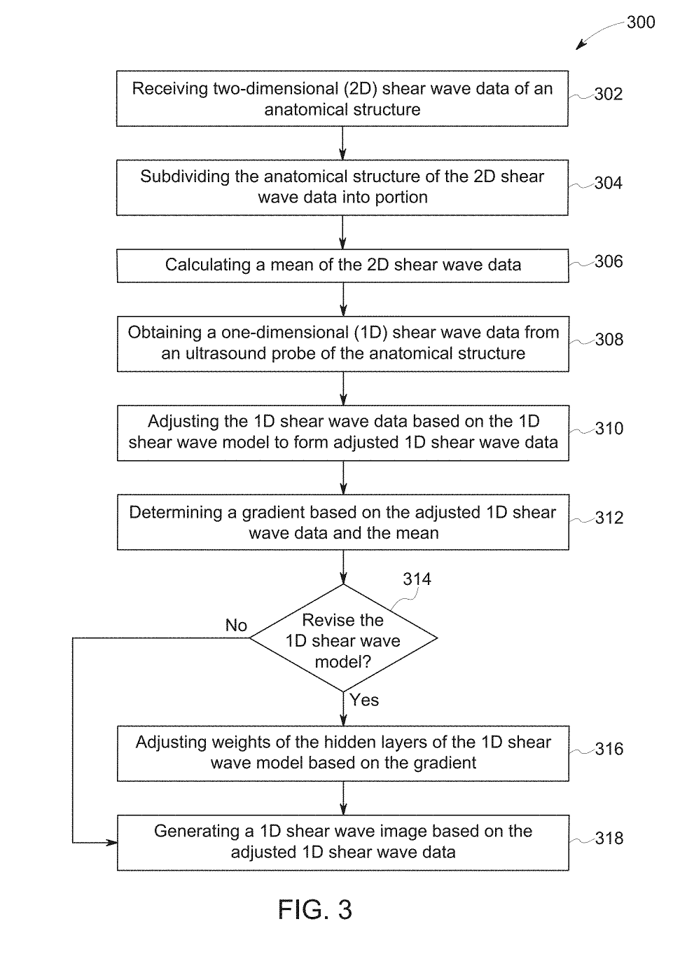

FIG. 3 illustrates a flow chart of an embodiment of a method 300 for correcting the 1D shear wave data 214 of an anatomical structure. The method 300, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. It may be noted that the steps described of the method 300 may be performed during the ultrasound exam in real-time. In various embodiments, portions, aspects, and/or variations of the method 300 may be used as one or more algorithms to direct hardware to perform one or more operations described herein.

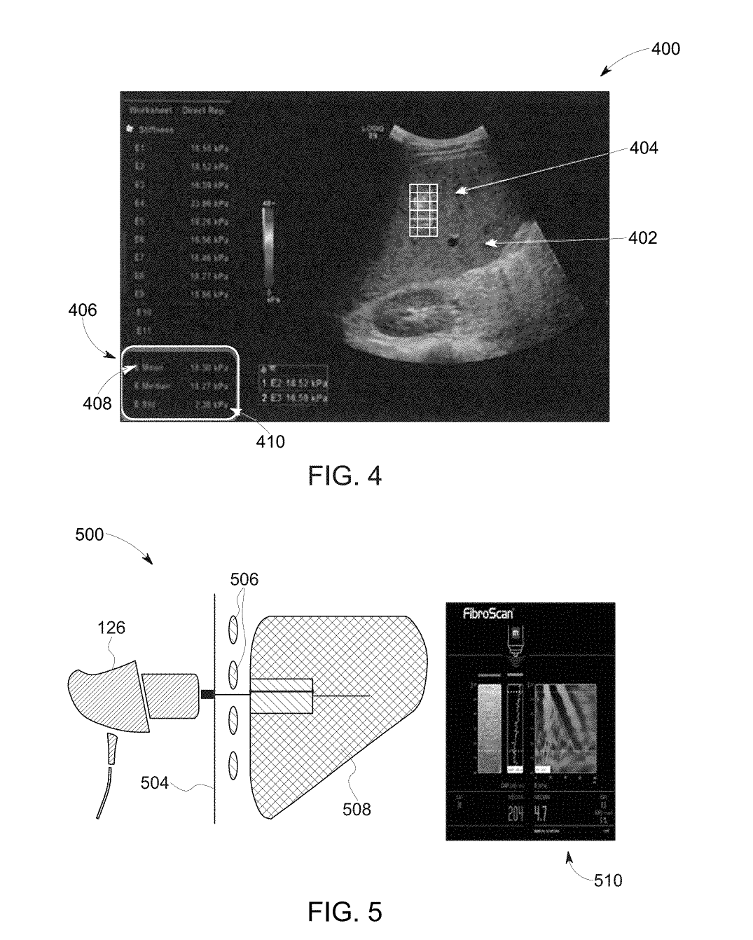

Beginning at 302, the controller circuit 102 receives 2D shear wave data 400 of an anatomical structure 402. FIG. 4 illustrates an embodiment of the 2D shear wave data 400. The anatomical structure 402 may represent an organ, such as a liver. The controller circuit 102 may receive the 2D shear wave data and/or the sets of 2D shear wave data 202 along the bi-directional and/or uni-directional communication link via the communication circuit 104. The 2D shear wave data and/or the sets of 2D shear wave data 202 can be acquired from one or more patients. The 2D shear wave data can be received from the remote server, the alternative medical imaging system, and/or the medical imaging system 100. For example, the controller circuit 102 activates the shear wave generator 111 by generating one or more pulses at a fixed frequency. The one or more pulses may represent tone bursts emitted from the shear wave generator 111 to the transducer array 112. The one or more pulses from the ultrasound probe 126 generates shear waves that traverse through the anatomical structure 402. As the shear waves are formed, the ultrasound probe 126 continually acquires ultrasound images (e.g., B-mode images). The controller circuit 102 compares the ultrasound images to identify changes in the shape and/or size of the anatomical structure 402. The changes in the shape and/or size of the anatomical structure 402 is indicative of a measured pressure exerted on the anatomical structure 402 by the shear waves. The measured pressure is utilized by the controller circuit 102 to form the 2D shear wave data 400.

At 304, the controller circuit 102 subdivides the anatomical structure 402 of the 2D shear wave data into portions 404. In connection with FIG. 4, the controller circuit 104 includes measurements of pressure and/or speed of the shear wave through the anatomical structure 404. The pressure is represented as different colors of pixels of the 2D shear wave data 400. The pressure can be indicative of a velocity of the shear wave traversing through the anatomical structure 402. The controller circuit 102 can calculate the velocity based on the acoustic impedance of the anatomical structure 202 and the measured pressure. For example, the acoustic impedance of the anatomical structure 202 may be stored in the memory 106. The anatomical structure 402 shown in FIG. 4, is a liver having an acoustic impedance of approximately 1.66 kg/m²s. The pressure indicated by the pixel is divided by the acoustic impedance to determine a velocity of the shear wave measured at the individual pixels and/or portions 404. In connection with FIG. 2, the portions 404 (e.g., the portions 203a-n) of the anatomical structure 402 are received by the input layer 206 of the correction model 204. The portions 404 represent different anatomical features of the anatomical structure 402.

At 306, the controller circuit 102 calculates a mean (e.g., arithmetic mean, geometric mean) 408 of the 2D shear wave data 400. For example, the mean 408 is indicative of an average pressure and/or speed of the 2D shear wave data 400. Additionally or alternatively, the 2D shear wave data 400 may be removed based on a standard deviation 410 of the pressure. For example, if the standard deviation 410 is below a predetermined threshold stored in the memory 106 the controller circuit 102 determines that the mean 408 of the 2D shear wave data 400 is accurate. The predetermined threshold may represent a magnitude (e.g., 3 kPA, 4 kPA). The standard deviation 410 is shown being approximately 2 kPA. The controller circuit 102 determines that the standard deviation 410 is below the predetermined threshold. In another example, if the standard deviation is above the predetermined threshold the controller circuit 102 determines that the 2D shear wave data 400 is corrupted and/or the mean 408 is not an accurate representation of the 2D shear wave data 400. For example, the pressure and/or speed of the mean 408 is not an accurate representation of the 2D shear wave data 400. The controller circuit 102 may request new 2D shear wave data 400 from the remote server, the alternative medical imaging system, and/or the medical imaging system 100 based on the standard deviation 410.

At 308, the controller circuit 102 obtains the 1D shear wave data 510 from the ultrasound probe 126 of the anatomical structure 508. Additionally or alternatively, the 1D shear wave data 510 may be stored in the memory 106. FIG. 5 illustrates an embodiment 500 for acquisition of the 1D shear wave data 510. The anatomical structure 508 represents an organ, such as a liver. For example, the controller circuit 102 activates the shear wave generator 111 by vibrating the ultrasound probe 126 The vibration of the ultrasound probe 126 generates one or more shear waves that traverse through ribs 506 into the anatomical structure 508. As shown in FIG. 5, the shear waves may extend to approximately 25-65 centimeters within the anatomical structure 508. When the one or more shear waves traverses through the anatomical structure 508, the ultrasound probe 126 continually acquires ultrasound images (e.g., M-mode images). The controller circuit 102 compares the ultrasound images to identify tissue displacement of the anatomical structure 508 as the one or more shear waves traverses through the anatomical structure 508. The tissue displacement is indicative of a speed exerted on the anatomical structure 508 by the one or more shear waves, which is stored in the memory 106. Additionally or alternatively, based on the speed of the shear wave a pressure (e.g., kPa) can be determined using the Young's modulus.At 310, the controller circuit 102 adjusts the 1D shear wave data 510 based on the correction model 204 to form the adjusted shear wave data 218 (FIG. 2). The controller circuit 102 adjusts a velocity and/or a pressure of the 1D shear wave data based on the correction model 204 to form the adjusted 1D shear wave data 218. The adjustment of the velocity and/or the pressure of the 1D shear wave data 510 is based on weights applied to the 1D shear wave data 510 from the hidden layers 208 of the correction model 204. For example, the controller circuit 102 executes the correction model 204 stored in the memory 106. The controller circuit 102 identifies an increase in a velocity and/or pressure of the pixels of the 1D shear wave data 510, which can represent fat and/or muscle content. The controller circuit 102 directs the correction model 204 representing different anatomical features (e.g., densities) of the anatomical structure 508. For example, the controller circuit 102 directs the 1D shear wave data 510 representing fat content to a first portion of the hidden layers 208. In another example, the controller circuit 102 directs the 1D shear wave data 510 representing muscle content to a second portion of the hidden layers 208. The hidden layers 208 adjust the 1D shear wave data 208 by adjusting a color and/or intensity, which represent velocity and/or pressure, of the pixels based on the weight matrixes of the first and second portions of the hidden layers 208.

At 312, the controller circuit 102 determines a gradient based on the adjusted 1D shear wave data 218 and the mean 216, 408. For example, the controller circuit 102 calculates the output error (e.g., loss) with respect to the weight matrixes of the hidden layers 208 based on the mean 216, 408 and the adjusted 1D shear wave data 218. The controller circuit 102 adjusts the 1D shear wave data 218 by the mean 216, 408 as shown in Equation 1. The controller circuit 102 calculates the gradient based on Equation 2.

At 314, the controller circuit 102 determines whether to revise the correction model 204. The controller circuit 102 revises the correction model 204 based on a loss (e.g., represented as the gradient) between the mean 216, 408 of the 2D shear wave data and the adjusted 1D shear wave data 218. For example, the controller circuit 102 determines a value and/or magnitude of the gradient. If the gradient is approximately zero (e.g., has an absolute value less than one), the controller circuit 102 determines that the correction model 204 does not need to be revised. Alternatively, if the gradient is not approximately zero the controller circuit 102 determines that the correction model 204 should be revised.

If the controller circuit 102 determines that the correction model 204 should be revised, then at 316, the controller circuit 102 adjusts the weights of the hidden layers 208 of the correction model 204 based on the gradient. For example, the controller circuit 102 shifts values of the weight matrixes of the hidden layers 208 based on the gradient. As shown in Equation 2, the controller circuit 102 adds/subtracts the gradient to adjust the weight matrixes and revise the correction model 204.

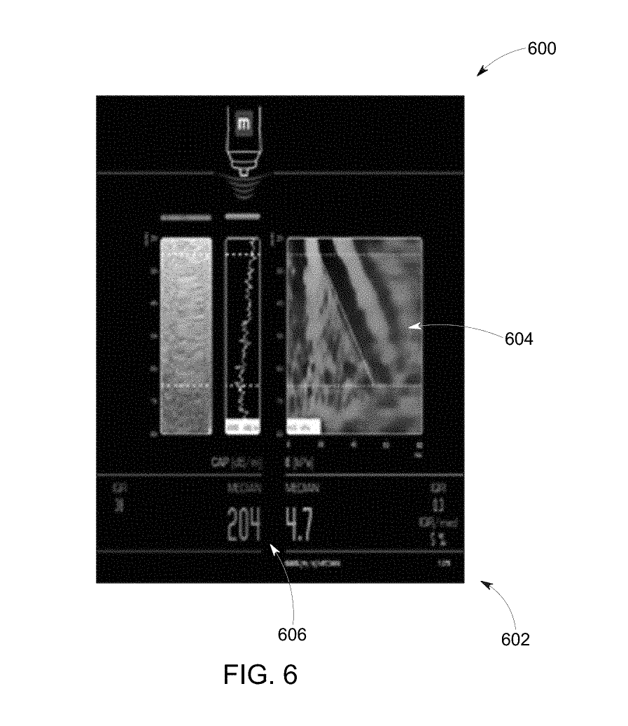

At 318, the controller circuit 102 generates a 1D shear wave image 602 based on the adjusted 1D shear wave data 218. FIG. 6 illustrates an embodiment 600 for generating the 1D shear wave image 602. For example, the 1D shear wave image 602 includes pixel information 604 representing a pressure as the shear wave traverses through the anatomical structure 508 (FIG. 5). Optionally, the 1D shear wave image 602 includes numerical information 606 representing a mean pressure and/or velocity of the shear wave traversing through the anatomical structure 508.

It may be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem,” “controller circuit,” “circuit,” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “controller circuit”.

The computer, subsystem, controller circuit, circuit execute a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer, subsystem, controller circuit, and/or circuit to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

1. A computer implemented method, comprising:
obtaining 1D shear wave data from an ultrasound probe of the anatomical structure;
adjusting a velocity or a pressure of the 1D shear wave data based on a correction model to form adjusted 1D shear wave data, wherein the adjustment of the velocity or the pressure of the 1D shear wave data is based on weights applied to the 1D shear wave data from hidden layers of the correction model; and
generating a 1D shear wave image based on the adjusted 1D shear wave data.

2. The computer implemented method of claim 1, wherein the obtaining operation includes vibrating the ultrasound probe from a shear wave generator, and identifying tissue displacement as the shear wave traverses through the anatomical structure, which forms the 1D shear wave data.

3. The computer implemented method of claim 1, further comprising receiving two-dimensional (2D) shear wave data of an anatomical structure; and
revising the correction model based on a loss between a mean of the 2D shear wave data and the adjusted 1D shear wave data;

4. The computer implemented method of claim 3, wherein the receiving operation includes generating a series of pulses from a shear wave generator, and identifying changes in a shape or size of the anatomical structure as the shear wave traverses through the anatomical structure, which forms the 2D shear wave data.

5. The computer implemented method of claim 3, wherein the revising operation includes adjusting weights of hidden layers of the correction model based on the loss.

6. The computer implemented method of claim 5, wherein the revising operation includes calculating a gradient based on the mean and the adjusted 1D shear wave data, weight factors of the correction model being adjusted based on the gradient.

7. The computer implemented method of claim 3, further comprising subdividing the anatomical structure of the 2D shear wave data into portions.

8. The computer implemented method of claim 1, further comprising identifying anatomical features of the 1D shear wave data that is indicative of a density of the anatomical structure.

9. The computer implemented method of claim 8, wherein the anatomical features is indicative of fat or muscle content of the anatomical structure.

10. The computer implemented method of claim 1, wherein the correction model includes a Stacked Denoise Autoencoder architecture, the correction model is configured to apply weights based on 2D shear wave data.

11. A medical imaging system comprising:
an ultrasound probe configured to acquire 1D shear wave data of an anatomical structure;
a communication circuit configured to receive two-dimensional (2D) shear wave data along a communication link from a remote server;
a display; and
a controller circuit configured to:
obtain the 1D shear wave data from an ultrasound probe of the anatomical structure;
adjust a velocity or a pressure of the 1D shear wave data based on a correction model to form adjusted 1D shear wave data, wherein the adjustment of the velocity or the pressure of the 1D shear wave data is based on weights applied to the 1D shear wave data from hidden layers of the correction model; and
generate a 1D shear wave image based on the adjusted 1D shear wave data on the display.

12. The medical imaging system of claim 11, further comprising a shear wave generator, wherein the controller circuit is configured to vibrate the ultrasound probe by the shear wave generator, and identify tissue displacement as the shear wave traverses through the anatomical structure to form the 1D shear wave data.

13. The medical imaging system of claim 11, wherein the controller circuit is configured to receive 2D shear wave data of a region of interest (anatomical structure) via the communication link, and revise a correction model based on a loss between a mean of the 2D shear wave data and the adjusted 1D shear wave data.

14. The medical imaging system of claim 13, further comprising a shear wave generator, wherein the controller circuit is configured to generate one or more pulses to form a series of pulses from the shear wave generator, and identify changes in a shape or size of the anatomical structure as the shear wave traverses through the anatomical structure to form the 2D shear wave data.

15. The medical imaging system of claim 13, wherein the controller circuit is configured to adjust weights of hidden layers of the correction model based on the loss.

16. The medical imaging system of claim 15, wherein the controller circuit is configured to calculate a gradient based on the mean and the adjusted 1D shear wave data, weight factors of the correction model being adjusted based on the gradient.

17. The medical imaging system of claim 13, wherein the controller circuit is configured subdivide the anatomical structure of the 2D shear wave data into portions.

18. The medical imaging system of claim 11, wherein the controller circuit is configured to identify anatomical features of the 1D shear wave data that is indicative of a density of the anatomical structure, wherein the anatomical features is indicative of fat or muscle content of the anatomical structure.

19. The medical imaging system of claim 11, wherein the correction model includes a plurality of neural layers based on a Stacked Denoise Autoencoder architecture, the correction model is configured to apply weights based on 2D shear wave data.

20. A tangible and non-transitory computer readable medium comprising one or more programmed instructions configured to direct one or more processors to:
obtain 1D shear wave data from an ultrasound probe of the anatomical structure;
adjust a velocity or a pressure of the 1D shear wave data based on a correction model to form adjusted 1D shear wave data, wherein the adjustment of the velocity or the pressure of the 1D shear wave data is based on weights applied to the 1D shear wave data from hidden layers of the correction model; and
generate a 1D shear wave image based on the adjusted 1D shear wave data.
